(19) United States
(12) Patent Application Publication
(10) Pub. No.: US 2004/0236420 A1
(43) Pub. Date: Nov. 25, 2004

(54) Artificial heart pump equipped with hydrodynamic bearing

(76) Inventors: Takashi Yamane, Ibaraki (JP); Yasushi Hisabe, Kanagawa (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/482,420

(22) PCT Filed: Jul. 12, 2002
(86) PCT No.: PCT/JP02/07131

(30) Foreign Application Priority Data
Jul. 12, 2001 (JP) 2001-211639

(51) Int. Cl. A61M 1/12; A61M 1/10

(52) U.S. Cl. 623/3.14; 415/900

(57) ABSTRACT

An artificial heart pump includes a casing (4, 15) having a blood inflow port (5) in its upper part, a blood outflow port (6) in its side surface part and a plurality of electromagnets (22) on its inner peripheral surface; a fixed shaft (17) projecting from the bottom surface of the casing and having thrust receptacles (18, 16) at its upper and lower end parts (12, 10), respectively; an impeller section (2) having a blood inflow section (3) in its center part and a blood outflow section (9) in its side surface part; an impeller support member (7) supporting the impeller section from below and having on its outer peripheral surface a plurality of permanent magnets (2) and in its center a hole part fitted on the fixed shaft to rotatably support the impeller section within the casing; a radial hydrodynamic bearing part formed between the inner peripheral surface of the hole part of the impeller support member and the outer peripheral surface of the fixed shaft; and thrust hydrodynamic bearings formed between the upper and lower end faces of the impeller support member and the thrust receptacles at the upper and lower end parts of the fixed shaft, respectively, whereby the impeller member is supported without contacting either the casing or the fixed shaft and rotates stably.

### TECHNICAL FIELD

This invention relates to an artificial heart pump used in place of or together with the heart of a living body and particularly to an artificial heart pump having impellers supported in the radial and thrust directions by hydrodynamic bearings.

### BACKGROUND ART

The Organ Transplant Law has come into effect also in Japan and allows heart transplants from brain-dead patients. For lack of brain-dead donors, however, it is the real state of affairs that the only way to save recipients still existing from death is to use an artificial heart. Studies on an artificial heart have been made for a long period of time and a large number of clinical demonstrations thereof have been reported. Artificial hearts include assist hearts inserted into a living body in parallel to the natural heart, with removal of the natural heart not accompanied, and total artificial hearts substituted for the natural heart and bonded. Almost all of these conventional artificial hearts are of an air-driven type requiring a controller to be installed at a patient's bedside. In recent years, however, assist hearts that can be embedded in the abdomen and electrically driven by a battery attached to a belt or rucksack have been developed. Though the artificial heart products available nowadays are used, from the standpoint of the size thereof, exclusively for patients of large physique, there have been used artificial hearts also suitable for at home remedy.

These artificial hearts are roughly divided in terms of pumping system into two types, namely, a pulsation flow type and a continuous flow type. The pulsation flow type adopts a system of sending a constant amount of blood out every one pulsation and, of assists hearts advanced in clinical application, there are those having year-basis actual use results. The continuous flow type adopts a system of using a rotary mechanism to continuously send blood out, with the amount of blood sent out relating not directly to the volume of a pump used, can be made small in size and is a promising one as an internally embedded type assist heart. According to some experiments with animals as regards the effect of no pulsation flow on a living body, their existence with no physiological defect has been reported. However, since it is recognized that the pulsation flow is preferable from the physiological point of view, the development of the continuous flow type pumps is progressing as an assist heart inserted with the natural heart remaining in a living body. The continuous flow type pumps induce centrifugal, axial-flow and rotary positive-displacement pumps. The present invention relates to an axial-flow pump of the continuous flow pumps.

As a continuous flow type artificial heart pump, one of the present inventors proposed a centrifugal pump for an artificial heart as shown in FIG. 3 (JP-A HEI 10-33664, U.S. Pat. No. 6,015,434. In the artificial heart pump as shown in FIG. 3, a centrifugal impeller 52 is supported by two bearings 56-58 and 55-60. A casing 57 is provided at the lower portion thereof with an impeller-driving device 61 in which a magnet 63 is rotated to rotation-drive magnets 54 embedded in the impeller. This allows blood to flow into the casing via an inflow port 64 formed at the upper part of the casing, and the blood can flow out of the casing via an outflow port formed around the lower part of the casing. Further, as means for rotating the impeller using the magnetic coupling as mentioned above, means adopting a direct-drive-system driving device that substitutes a group of electromagnets for a movable portion 66 has been developed.

In the proposed artificial heart pump, the impeller is supported in the radial direction by means of repulsive force between a magnet 56 provided at the outer periphery of an impeller cylindrical portion 51 and a support magnet 58 disposed at the opposed position and in the thrust direction by means of fitting between a pivot shaft 55 projecting from the bottom surface of the impeller 53 and a pivot receptacle 60 provided at the center of the bottom plate 59 of the casing. The impeller thus supported is driven using an impeller-driving device 61 disposed on the lower portion of the casing and rotating a magnet 63 facing magnets 54 provided on the lower portion of the impeller, or rotating the magnet 63 constituted by an electromagnet in accordance with a direct drive system.

However, the aforementioned impeller-supporting system requires fixing multiple magnets to the impeller and casing and taking multiple steps to produce a pump and makes the impeller heavy in weight owing to the fixed magnets. In addition, since the pivot shaft and receptacle are friction-slid against each other, and, through use thereof over a long period of time, friction powder are gradually accumulated at the sliding contact surface to possibly induce a cause of shortening the service life of the pump and a cause of thrombosis due to blood stagnation at the bearing portion.

The present invention has been accomplished based on the findings mentioned above and its object is to provide an artificial heart pump that is lightweight as compared with a conventional artificial heart pump, eliminates accumulation of friction powder resulting from friction slide and suppresses occurrence of blood stagnation at a bearing portion.

### DISCLOSURE OF THE INVENTION

An a heart pump according to the present invention comprises a casing having a blood inflow port in an upper part, a blood outflow port in a side surface part and a plurality of electromagnets on an inner peripheral surface; a fixed shaft projecting from a bottom surface of the casing and having thrust receptacles at upper and lower end parts, respectively; an impeller section disposed inside the casing, having a blood inflow section in a center part and a blood outflow section in a side surface part, and comprising a plurality of impellers; an impeller support member supporting the impeller section from below and having in a center a hole part rotatably fitted on the fixed shaft to rotatably support the impeller section; a plurality of permanent magnets provided on an outer peripheral section of the impeller support member at positions facing the plurality of electromagnets on the inner peripheral surface of the casing; a radial hydrodynamic bearing formed between an inner peripheral surface of the hole part of the impeller support member and an outer peripheral surface of the fixed shaft; and a trust hydrodynamic bearing formed between upper and lower end faces of the impeller support member and the thrust receptacles at the upper and lower end part of the fixed shaft

In the artificial heart pump according to the present invention, the impeller support member is provided with a plurality of thrust hydrodynamic pressure generation grooves at positions respectively facing the thrust receptacles at the upper and lower end parts of the fixed shaft, and the fixed shaft is provided a lower outer periphery with a plurality of radial hydrodynamic pressure generation grooves to form a first thrust hydrodynamic bearing part, the radial hydrodynamic bearing and a second thrust hydrodynamic bearing part in this order.

In the artificial heart pump, the thrust generation grooves facing the thrust receptacle at the lower end part of the fixed shaft have a pump-in type spiral pattern, and the thrust generation grooves facing the thrust receptacle at the upper end part have a pump-out type spiral pattern.

As described above, the artificial heart pump according to the present invention has the radial hydrodynamic bearing formed between the cylindrical inner surface of the impeller support member and the outer peripheral surface of the fixed shaft and also has the thrust hydrodynamic bearings formed respectively between the upper and lower end faces of the impeller support member and the thrust receptacles formed at the upper and lower end parts of the fixed shaft. Therefore, the impeller section is retained by these bearings and rotated in a floating state in the radial and thrust directions, and the blood circulates the first thrust hydrodynamic bearing part, radial hydrodynamic bearing part and second thrust bearing part in this order.

Therefore, it is possible to provide an artificial heart pump that is lightweight in consequence of the use of a small number of magnets and, because the impeller section is rotated in a floating state, eliminates friction powder and occurrence of blood stagnation at the bearing parts.

### BRIEF DESCRIPTION OF THE DRAWINGS

FIG. 1 is a cross section showing one embodiment of an artificial heart pump according to the present invention.

FIGS. 2(a), 2(b) and 2(c) are explanatory views showing the configuration of bearings of the artificial heart pump shown in FIG. 1.

FIG.3 is a cross section showing a prior art artificial heart pump.

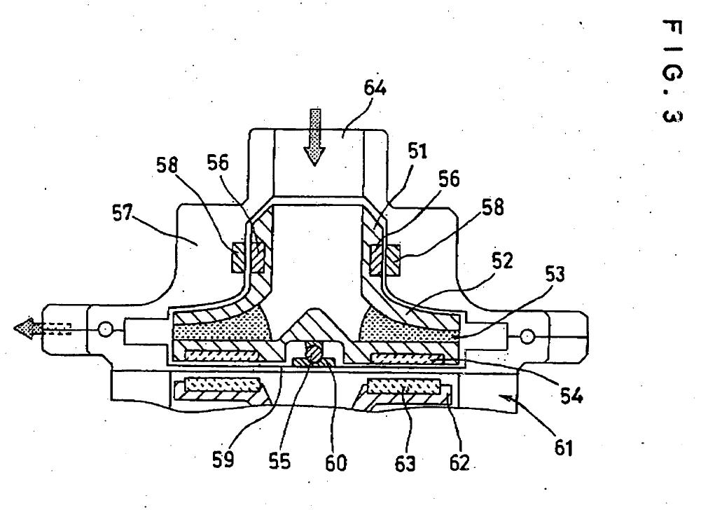

### BEST MODE FOR CARRYING OUT THE INVENTION

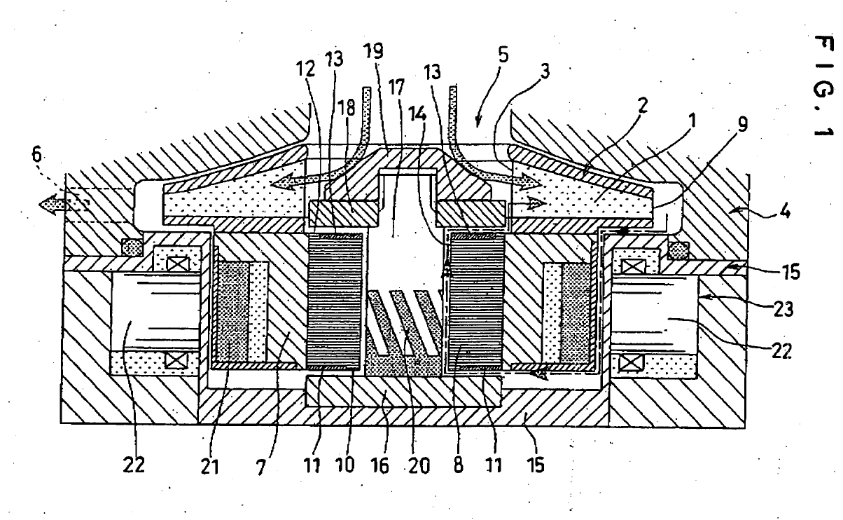

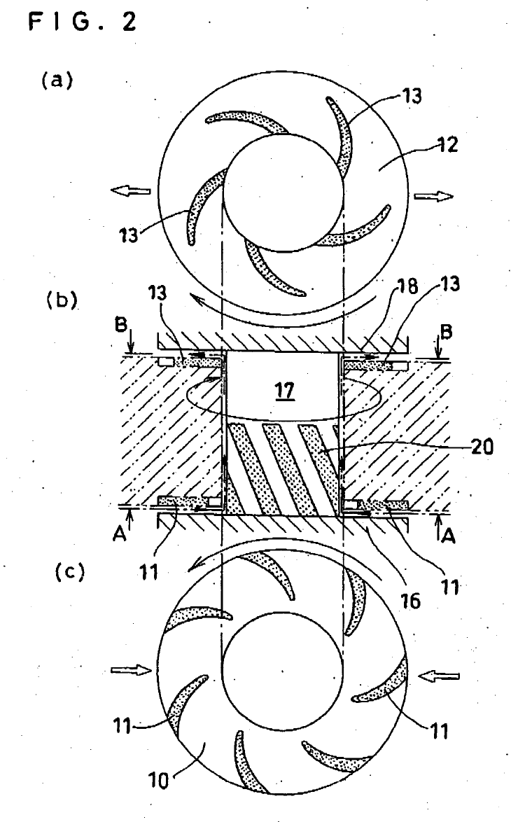

An artificial heart pump according to the present invention will be described with reference to the drawings. FIG. 1 is a cross section showing one embodiment of the artificial heart pump in the present invention, and FIG. 2 is an explanatory view showing the configuration of hydrodynamic bearings. In FIG. 1, an impeller section 2 equipped with a plurality of impellers 1 extending radially within an upper casing 4 has its center part opened to define a blood inflow section 3, sucks blood in from a cylindrical blood inflow port 5 formed in the upper casing 4 when rotating the impellers 1 as described later, and discharges the sucked blood out from a blood outflow port 6 formed in the side surface of the upper casing 4.

The impeller section 2 is supported on a cylindrical impeller support member 7 that is provided at its center integrally with a cylindrical bearing member 8. The bearing member 8 that is a part of the impeller support member 7 has a lower end face 10 formed therein with lower thrust hydrodynamic pressure generation grooves 11 having a pump-in type spiral pattern as shown in FIG. 2(c) and has an upper end face 12 formed therein with upper thrust hydrodynamic pressure generation grooves 13 having a pump-out type spiral pattern as shown in FIG. 2(a).

A hole part formed in the center of the cylindrical bearing member 8 is fitted on a fixed shaft 17 projecting from the upper surface of a lower thrust receptacle 16 fixed to a lower casing 15 to form a cylindrical passageway section 14 of a predetermined width. A radial hydrodynamic bearing part that rotatably supports the impellers 1 and impeller support member 7 is thus constituted. The lower thrust receptacle 16 is disposed to face and separate by a predetermined interval from the lower end face 10 of the bearing member 8 having the lower thrust hydrodynamic pressure generation grooves 11. The upper thrust receptacle 18 is fixed to the upper part of the fixed shaft 17 by means of a fixing member 19, leaving a predetermined interval relative to the upper end face 12 of the bearing member 8 having the upper thrust hydrodynamic pressure generation grooves 13. In addition, the fixed shaft 17 is formed in its lower outer periphery with inclined grooves 20 for generation of radial hydrodynamic pressure.

The impeller support member 7 is provided on its outer periphery with a plurality of permanent magnets 21 disposed at predetermined intervals. The lower casing 15 is provided on its outer periphery with a plurality of electromagnets 22 disposed to face the permanent magnets 21. The electromagnets 22 with alternating polarities, when applying electricity thereto, constitute a direct drive type motor that is an impeller-driving device 23. When setting a motor flux to direct in the diametrical direction, it is possible to avoid exerting an excess load onto a thrust hydrodynamic bearing.

With the above configuration, by applying electricity to the electromagnets 22 with alternating polarities and rotating the impeller support member 7, the impeller section 2 equipped with the impellers 1 is rotated to suck blood in from the blood inflow port 5, pressurize the sucked blood during a course from the blood inflow section 3 to a blood outflow section 9 of the impellers 1 and discharge the pressurized blood out from the blood outflow port 6.

Part of the pressurized blood from the blood outflow section 9 formed in the side part of the impellers 1 circulates a flow path comprising, as shown by a one-dot-line arrow in the figure, the gap between the lower surface of the impeller section 2 and the upper surface the lower casing 15, gap between the outer peripheral surface the impeller support member 7 and the facing cylindrical inner wall surface of the lower casing 15, thrust hydrodynamic bearing part formed between the upper surface of the lower thrust receptacle 16 and the lower end face 10 of the bearing member 8 that is a part of the impeller support member 7, radial hydrodynamic bearing part comprising the cylindrical passageway section 14 formed between the outer peripheral surface of the fixed shaft 17 and the inner circumferential surface of the hole part 14 of the bearing member 8, thrust hydrodynamic bearing part formed between the upper end face of the bearing member 8 and the lower surface of the upper thrust receptacle 18, and the low pressure side of the blood inflow section 3 of the impeller section 2.

In the flow path between the upper section of the lower receptacle 16 and the lower end face 10 of the bearing member 8, ie. on the lower surface of the impeller support member 7 in the illustrated embodiment, the lower-side thrust hydrodynamic pressure generation grooves 11 having the pump-in type spiral pattern are formed. There, as shown in FIG. 2(c), for example, the blood flowing along the flow path is sucked in from the outer peripheral side of the lower-side thrust hydrodynamic pressure generation grooves 11 and discharged out toward the inner peripheral side thereof. The hydrodynamic pressure generated at this time supports the force of the entire impeller section in the downward thrust direction.

The inner peripheral side of the lower-side thrust hydrodynamic pressure generation grooves 11 communicates with the cylindrical passageway section 14 formed between the outer peripheral surface of the fixed shaft 17 and the cylindrical inner peripheral surface of the bearing member 8. In the passageway section, i.e. in the outer periphery of the fixed shaft 17 in the illustrated embodiment, the plurality of inclined hydrodynamic pressure generation grooves 20 are formed. Therefore, as shown in FIG. 2(b), the blood is sucked in from the lower end side o the fixed shaft and discharged out toward the upper end side thereof. The hydrodynamic pressure generated at this time supports the force of the entire impeller section in the radial direction.

The flow of blood thus discharged out toward the upper end side of the fixed shaft 17 is sucked in from the inner peripheral side of the upper-side thrust hydrodynamic pressure generation grooves 13 and charged out toward the outer peripheral side thereof because the upper-side hydrodynamic pressure generation groove 13 having the pump-out type spiral pattern are formed in the gap between the upper end face 12 of the bearing member 8 and the lower surface of the upper thrust receptacle 18, ie. on the upper surface of the impeller support member 7 in the illustrated embodiment

The flow of blood thus discharged out is sucked in toward the blood inflow section 3 of the impellers 1, mixed with new blood sucked in from the blood inflow port 5, pressurized by the impellers 1 and discharged out The hydrodynamic force generated at this time supports the force of the entire impeller section in the upward thrust direction, and in conjunction with the hydrodynamic pressure supporting the force in the downward thrust direction by the lower-side thrust hydrodynamic pressure generation grooves 11, supports the entire impeller section in the vertical direction. Thus, the impeller section is retained in the predetermined floating state.

By means of the constitution and function of these bearings, the impeller section can stably rotates without contacting the upper casing 4, lower casing 15, center fixed shaft 13, etc. surrounding the impeller section. In addition, since the fluid generating hydrodynamic pressure at the hydrodynamic bearing parts that support the impeller section is a liquid and highly viscous blood, the impeller section can infallibly be supported. Furthermore, since the fluid is the fluid circulating in the flow path from the high-pressure side of the outflow section of the impeller to the low-pressure side of the inflow section thereof and since the hydrodynamic pressure generation grooves are formed so that the fluid can flow along the flow pass, the hydrodynamic pressure generation fluid can stably flow. In this connection, the impeller section can infallibly be supported at the bearing parts. Moreover, since the blood can stably flows at the bearing parts without staying there, blood stagnation can be prevented from occurring.

In the embodiment shown in FIG. 1, since the bearing member 8 is disposed at the center side of the impeller support member 7 that supports the impeller section 2 and the permanent magnets are disposed at the outer peripheral side thereof, the impeller section 2 can stably be rotated and the artificial heart pump can be reduced in height and made compact as a whole to provide an artificial heart pump suitable as an internally embedded type.

While the hydrodynamic pressure generation grooves 20 are formed in the outer periphery of the fixed shaft 17 fixed at the center as the radial hydrodynamic bearing in the forgoing embodiment, these may be formed in the inner peripheral of the bearing member 8.

### Industrial Applicability

With the configuration of the present invention as described above, it is possible to provide an artificial heart pump that is lightweight as compared with a prior art pump using a magnetic bearing and, as compared with a prior art pump using a pivot bearing, eliminates generation of friction powder and occurrence of blood stagnation at the bearing parts.

1. An artificial heart pump comprising:
a casing (4, 15) having a blood inflow port (5) in an upper part, a blood outflow port (6) in a side source part and a plurality of electromagnets (22) on an inner peripheral surface;
a fixed shaft (17) projecting from a bottom surface of the casing and having thrust receptacles (18, 16) at upper and lower end parts (12, 10), respectively;
an impeller section (2) used inside the casing, having a blood inflow section (3) in a center part and a blood outflow section (9) in a side surface part, and comprising a plurality of impellers (1);
an impeller support member (7) supporting the impeller section from below and having in a center a hole part fitted on the fixed shaft to rotatably support the impeller section;
a plurality of permanent magnets (21) provided on an outer peripheral surface of the impeller support member at positions facing the plurality of electromagnets (22) on the inner peripheral surface of the casing;
a radial hydrodynamic bearing formed between an inner peripheral surface of the hole part of the impeller support member and an outer peripheral surface of the fixed shaft; and
a thrust hydrodynamic bearing formed between upper and lower end faces of the impeller support member and the thrust receptacles at the upper and lower end parts of the fixed shaft.

2. The artificial heart pump according to claim 1, wherein the impeller support member is provided with a plurality of thrust hydrodynamic pressure generation grooves (13, 11) at positions respectively facing the thrust receptacles (18, 16) at the upper and lower end parts (12, 10) of the fixed shaft, and the fixed shaft is provided a lower outer periphery with a plurality of radial hydrodynamic pressure generation grooves (20) to form a first thrust hydrodynamic bearing part, the radial hydrodynamic bearing and a second thrust hydrodynamic bearing part in this order.

3. The artificial heart pump according to claim 2, wherein the thrust generation grooves (11) facing the thrust receptacle at the lower end part of the fixed shaft have a pump-in type spiral pattern to suck blood in from an outer peripheral side of the grooves and discharge the sucked blood to an inner peripheral side thereof, and the thrust generation grooves (13) facing the thrust receptacle at the upper end part have a pump-out type spiral pattern to suck blood in from an outer peripheral side of the grooves and discharge the sucked blood to an inner peripheral side thereof.
